(19) United States
(12) Patent Application Publication
(10) Pub. No.: US 2017/0290975 A1
(43) Pub. Date: Oct. 12, 2017

(54) AUTOMATIC INJECTION DEVICE HAVING A DRIVE SYSTEM WITH A SHAPE MEMORY SPRING

(71) Applicant: FLEXTRONICS AP, LLC, San Jose, CA (US)

(72) Inventors: Eyal BARMAIMON, Haifa (IL); Lior SHTRAM, Tel Aviv (IL); Boaz KALISCH, Haifa (IL); Ronny BELLAN, Nazareth (IL); Nadav CHOEN, Haifa (IL); Shai FINKMAN, Haifa (IL); Nadav MEROM, Haifa (IL)

(73) Assignee: FLEXTRONICS AP, LLC, San Jose, CA (US)

(21) Appl. No.: 15/436,529

(22) Filed: Feb. 17, 2017

Related U.S. Application Data
(60) Provisional application No. 62/319,258, filed on Apr. 6, 2016.

(51) Int. Cl. A61M 5/145; A61M 5/142

(52) U.S. Cl. CPC A61M 5/1454; A61M 5/14248; A61M 2205/0266

(57) ABSTRACT

An automatic injection device has an insertion needle configured to be inserted into a patient and a drug container which contains a pharmaceutical product and includes a plunger. The automatic injection device also has a fluid path which fluidly connects the drug container to the insertion needle, and a drive system configured to cause linear movement of the plunger to force the pharmaceutical product into the fluid path. The drive system has a movable element. The movable element has a shape memory alloy and is configured to change shape to move the plunger.

### PRIORITY CLAIM

This application claims priority under 35 U.S.C. §119 to U.S. Provisional Application No. 62/319,258, filed on Apr. 6, 2016 which is expressly incorporated by reference herein in its entirety.

### FIELD OF INVENTION

The present disclosure relates to an automatic injection device, and, in particular, to an automatic injection system having a drive system with a shape memory spring.

### BACKGROUND

Injection devices are used to deliver pharmaceutical products such as biologics and medications to a patient (i.e., a person or animal). A syringe and needle is an example of a widely-used injection device. This basic system typically involves a person manually moving a plunger portion of the syringe to force the pharmaceutical product through the needle and into the patient. Other injection devices have been developed to deliver pharmaceutical products automatically at the touch of a button or the actuation of a switch. These devices are advantageous in that they allow a patient to more easily self-administer the pharmaceutical product. Moreover, some automatic injection devices allow for slow or periodic delivery of the pharmaceutical product as needed, which is typical procedure for patients dependent on insulin injections, for example.

However, there is a need for automatic injection devices to provide injection control in compact device such that the device is easy to handle and discrete for a patient who may wear the device for an extended period of time. Moreover, the elements of the device should be configured for easy replacement of the drug container when the previous container is empty while minimizing the risk of contamination of sterile components.

The present disclosure is direction to an automatic injection device which addresses these needs and the associated problems of the prior art.

### SUMMARY

In one aspect, the present disclosure is directed to an automatic injection device. The automatic injection device includes an insertion needle configured to be inserted into a patient and a drug container which contains a pharmaceutical product and includes a plunger. The automatic injection device also includes a fluid path which fluidly connects the drug container to the insertion needle, and a drive system configured to cause linear movement of the plunger to force the pharmaceutical product into the fluid path. The drive system includes a movable element. The movable element includes a shape memory alloy and is configured to change shape to move the plunger.

In another aspect, the present disclosure is directed to a product for use in an automatic injection device. The product includes a drug container configured to contain a pharmaceutical product and including a first longitudinal end and a second longitudinal end. The product also includes a plunger in the drug container configured to move in a linear direction from the first longitudinal end toward the second longitudinal end. The product additionally includes a movable element formed of a shape memory alloy in the drug container. The movable element is configured to move the plunger in the linear direction based on the shape memory properties of the movable element.

In yet another aspect, the present disclosure is directed to a cartridge for an automatic injection device. The cartridge includes a space for receiving a drug container which contains a pharmaceutical product, and a drive system including a driving element and a movable element, the movable element including a coil spring made from a shape memory alloy and being configured to linearly extend or contract based on the shape memory properties of the movable element in the space for receiving the drug container.

### BRIEF DESCRIPTION OF THE DRAWING(S)

FIG. 1 is a side view illustration of an exemplary automatic injection device positioned on a patient;

FIG. 2 is a schematic illustration of the components of an automatic injection device consistent with disclosed embodiments;

FIG. 3 is a perspective view of a top side of the automatic injection device;

FIG. 4 is a perspective view of a bottom side of the automatic injection device;

FIG. 5 is a perspective view of the internal components according to an embodiment of the automatic injection device;

FIG. 6 is a perspective view of a separated housing and cartridge according to an embodiment of the automatic injection device;

FIG. 7 is a perspective view of the drive system according to a first embodiment including a movable member in a first position;

FIG. 8 is a perspective view of the drive system according to the first embodiment including the movable member in a second position;

FIG. 9 is an exploded view of a movable member of a drive system of the automatic injection device according to another embodiment;

FIG. 10 is a perspective view of the drive system including the movable member of FIG. 9 in a first position; and

FIG. 11 is a perspective view of the drive system including the movable member of FIG. 9 in a second position.

### DETAILED DESCRIPTION OF THE PREFERRED EMBODIMENT(S)

Disclosed embodiments pertain to a drive system for an automatic injection device. The drive system provides an input force to move an element of the automatic injection device to control delivery of a pharmaceutical product to a patient. For example, the drive system may be configured to control movement of a plunger placed in a drug container. Further movement of the plunger inside the drug container pushes the pharmaceutical product through a fluid path and to a insertion needle which connects to the patient. This controlled movement of the plunger allows for metered delivery of the pharmaceutical product according to desired parameters.

The disclosed drive system includes features which allow for precise control over plunger movement while providing a small form factor which allows the automatic injection device to be compact. The disclosed drive system utilizes a shape memory spring in order to cause movement of the plunger. The shape memory spring may be positioned inside or outside of the drug container and may be used in conjunction with magnetic elements to cause movement of the plunger. In some embodiments, the shape memory spring is pre-configured with properties which cause the spring to move to assume a “memorized” shape. In returning to this shape, the spring can push the plunger. Because the movement is based on shape memory properties, the force applied to the plunger is substantially constant (as opposed to a conventional spring which applies a variable force). This allows for specific control of the movement of the plunger with a passive driving device.

In some embodiments, the shape memory spring is connected to a driving element which provides an input to change a shape of the shape memory spring, which is formed of a shape memory alloy. This change in shape causes movement of the plunger and thus movement of the pharmaceutical product out of the drug container (e.g., and into a patient through a fluid path).

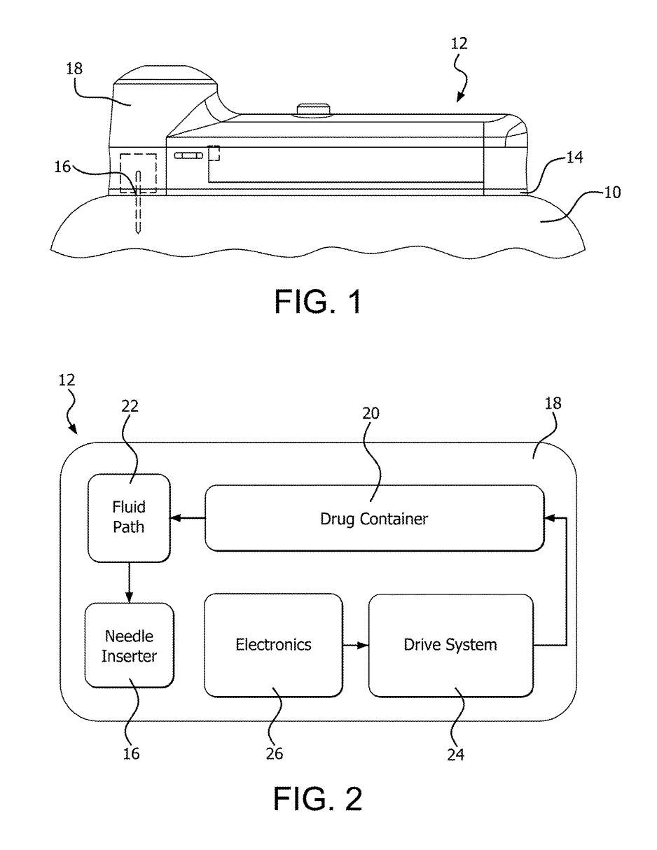

FIG. 1 is a depiction of an exemplary embodiment of an automatic injection device 12 on a patient 10. The patient 10 is not limited and can be any organism which may receive an injection. The device 12 is configured to deliver a pharmaceutical product to the patient 10 automatically. This means that the device 12 is controlling the injection in some way such that the system differs from other injection systems where manual input alone causes the injection (i.e., a conventional syringe and needle system or other available systems). The automatic aspect of the device 12 may additionally or alternatively relate to the duration of the injection, controlled injection intervals, a delay between input and injection, etc.

The device 12 includes a base 14 that contacts the patient's skin. The device 12 includes a insertion needle 16 which enters the patient 12 to deliver a pharmaceutical product, such as insulin, to the patient. An example of an automatic injection device which includes many of the features that may be incorporated into the device 12 is described in WO 2017/007952, which is herein incorporated by reference. However, it should be understood that the device 12 is not limited to the automatic injection device described therein or the exemplary embodiments described below. An automatic injection device consistent with this disclosure may include some of the features described herein but is not limited thereto. For example, a disclosed automatic injection device may resemble a syringe and needle system or other injection system which is adapted for automatic injections via the features described herein.

FIG. 2 is a schematic illustration of the automatic injection device 12 and the basic features of the device 12 which facilitate automatic injections of a pharmaceutical product into the patient 10. The device 12 preferably includes a housing 18 which holds at least some of the features of the device 12. In addition to the insertion needle 16, these features preferably include a drug container 20, a fluid path 22, a drive system 24, and electronics 26. These features work in conjunction with each other to automatically deliver a pharmaceutical product to the patient 10 according to desired parameters. In some embodiments, the drive system 24 may be a passive system which does not require electronics 26.

The drug container 20 is a containing element which supplies the pharmaceutical product. The drug container 20 may be a vial, syringe, or the like and includes a space for containing the pharmaceutical product, which is not limited to a particular substance. The pharmaceutical product may be any substance which is considered one or more of a drug, biologic, medication, or placebo, for example. The drug container 20 is preferably a hollow cylindrical tube which receives the pharmaceutical product. However, it should be understood that other configurations are possible.

In the device 12, the drug container 20 is fluidly connected to the insertion needle 16 by the fluid path 22. The fluid path 22 may be a physical connecting channel which serves as a conduit for delivering the pharmaceutical product from the drug container 20 to the insertion needle 16 and ultimately into the patient 10. The fluid path 22 may include additional structure, including actuating mechanisms which initiate delivery of the pharmaceutical product and/or control mechanisms which meter an amount of product which is delivered to the patient 10 at any particular time. The fluid path 22 may include an element or mechanism which is configured to establish the connecting channel, such as a puncturing needle or the like. The fluid path 22 may be associated with a start button or control switch or even the electronics 26 which control an element of the fluid path 22 (e.g., a valve) in order to start or stop the delivery of the pharmaceutical product. It should be understood, however, that the fluid path 22 may be a passive system component in at least some embodiments.

The drive system 24 and/or electronics 26 provide the automatic aspect of the injections via device 12. For example, the drive system 24 is a mechanical system which physically moves an element of the device 12 to move the pharmaceutical product from the drug container 20 to into the fluid path 22 and ultimately into the patient 10. For example, the drive system 24 may be configured to move a plunger positioned inside the drug container 20 to force the pharmaceutical product out of the drug container 20. The drive system 24 includes magnetic elements, as will be described in more detail herein. The electronics 26 include features such as control circuitry, processing devices, memory, I/O devices, etc. and are configured to control the drive system 24 such that the pharmaceutical product is delivered according to desired parameters. For example, the electronics may translate an input signal and provide a signal to the drive system 24 to move a plunger inside the drug container 20 by a selected amount.

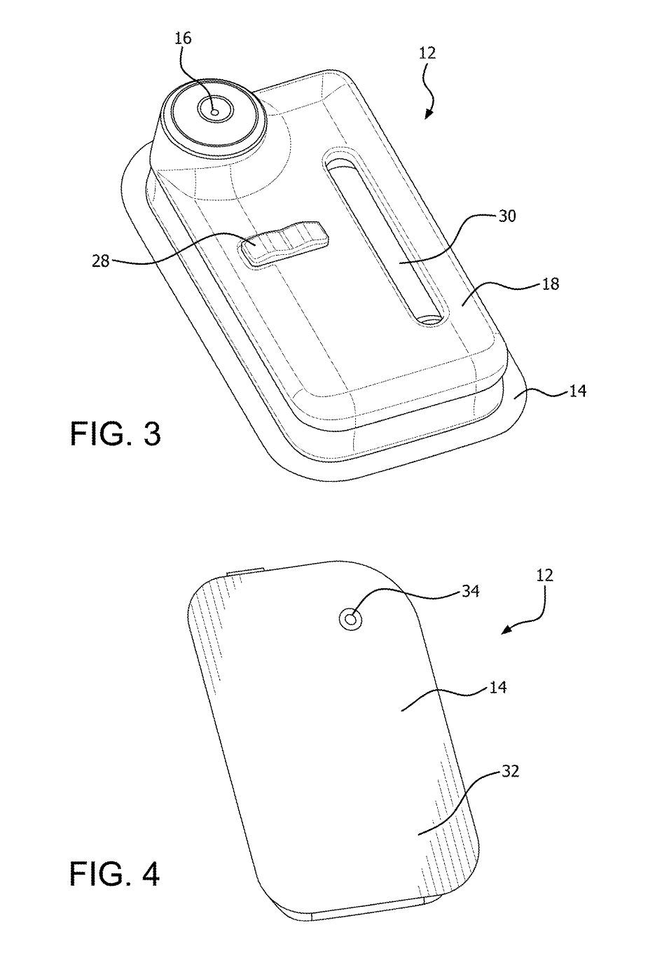

FIGS. 3 and 4 further illustrate an exemplary embodiment of the automatic injection system 12. FIG. 3 illustrates a first side of the device 12, including the housing 18 which rests on top of the base 14. The housing 18 is illustrated as being rectangular, but can include any shape. The housing 18 may include optional features such as at least one control switch 28 which provides an input signal to the electronics 26 and/or a window 30 which provides a view of the drug container 20 and thus the current fluid level.

FIG. 4 illustrates a second side of the device 12 including a bottom surface of the base 14. The bottom surface 32 includes an opening 34 for receiving the insertion needle 16 therethrough. In use, the device 12 is placed against the patient 10 with the bottom surface 32 of the base 14 against the skin. An injection needle extends through the opening 34 and into the patient 10 to deliver the pharmaceutical product. The bottom surface 32 may include an adhesive material thereon to adhere the device 12 to the patient 10 for either a short or long period of time, depending on the particular use of the device 12.

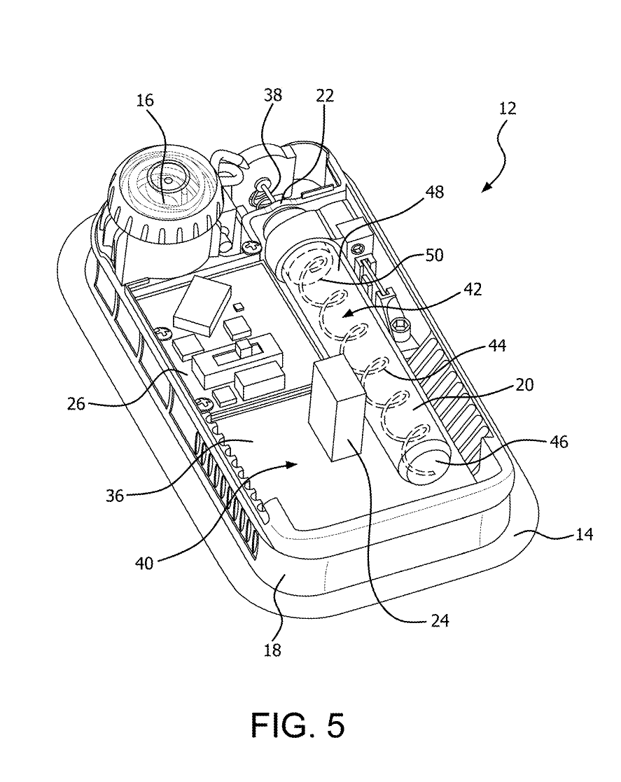

FIG. 5 illustrates the device 12 with the top portion of the housing 18 removed such that the internal features are shown in an enclosed space 36 formed by a lower portion of the housing 18 and the base 14. The device 12 includes exemplary embodiments of the insertion needle 16, the drug container 20, the fluid path 22, the drive system 24, and the electronics 26. The enclosed space 36 houses at least the drug container 20, drive system 24, and electronics 26 such that the elements are positioned inside of the housing 18.

The fluid path 22 in this embodiment includes a connector 38 which physically connects to the drug container 20 to establish a connection channel between the interior of the drug container 20 and the insertion needle 16. In the illustrated embodiment, the insertion needle 16 is positioned perpendicular to the drug container 20 such that the path of the pharmaceutical product is to travel out of the drug container 20 and laterally into the area of the insertion needle 16 via the fluid path 22. The pharmaceutical product subsequently travels vertically downward through the insertion needle 16 and into the patient 10. This configuration is exemplary, however, and disclosed embodiments are not limited thereto. In other embodiments, the insertion needle 16 may be aligned in the same direction as the drug container 20 and/or the fluid path 22.

The drive system 24, in some embodiments, includes a driving element 40 and a movable element 42. The driving element 40 is preferably a device which applies an input parameter to the movable element 42 to cause a change in the movable element 42. The movable element 42 is preferably a coil spring 44 made of a shape memory alloy. In some embodiments, the driving element 40 is omitted from the device 12. For example, the movable element 42 may be configured to move based on its own properties, such as spring and shape memory properties. In some instances, the driving element 40 may apply an input parameter to the spring 44 prior to the spring 44 being installed in the device 12, such as to cause the spring 44 to assume a particular shape.

Shape memory alloys, such as alloys of copper-nickel-aluminum or nickel-titanium, are metallic materials which change in shape when an input parameter is applied, such as heat or electric current. These changes occur due to a transition in the crystalline structure of the material, such as conversion between austenite and martensite. Shape memory alloys may include different shape configurations which occur under different conditions. For example, a shape memory alloy may have a low temperature shape and a high temperature shape. Application of heat (or current) to a shape memory alloy in its low temperature shape causes the material to assume its high temperature shape. In some materials, subsequent lowering of the temperature (or removal of current) of the material causes the shape memory alloy to return to the low temperature shape.

The shape memory alloy may elongate when changing between the different “memorized” shapes. With the coil spring 44 of the present disclosure being formed from a shape memory alloy, a change in temperature and/or current will cause the spring 44 to longitudinally extend or retract from its current position. Similarly, a change in shape (e.g., compression of a spring shape) may also cause the shape memory alloy to return to a “memorized” shape (e.g., an extended shape). This movement may appear like and be influenced by a spring-biasing properties, but includes shape memory properties which contribute to the change. This linear movement of the spring 44 (either through passive or driven change in shape) can be used to force a pharmaceutical product out of the drug container 20, as will be further described.

In embodiments of the device 12 which only include the movable element 42 (i.e., and omit the driving element 40), the movable element 42 is preferably formed as the coil spring 44 with shape memory properties. The coil spring 44 may also include biasing properties, similar to a conventional coil spring. In these embodiments, the spring 44 is preferably formed such that it changes shape by extending (similar to a conventional spring). However ,because the spring 44 is a shape memory alloy formed according to selected conditions, the spring 44 will apply a substantially constant force as it extends (unlike a conventional spring which applies a variable force as its length changes).

In one example, the coil spring 44 is made of a shape memory alloy and is shaped or compressed to a relatively short effective length. Due to the configuration of the coil spring 44 (e.g., previous application of an input parameter), the spring 44 is configured to extend into a longer effective length, thereby pushing anything in contact with a moving end of the spring 44. This change may occur under selected conditions, such as when the spring 44 is compressed at room temperature. It should be understood, however, that the change may also occur based on an input parameter, such as heat or current received from the driving element 40 (in embodiments which included the driving element 40).

When included, the driving element 40 is a device which applies an input parameter to the spring 44 to cause the spring to extend or retract in a linear direction. For example, the driving element 40 may be a heating element configured to heat or cool the spring 44 and/or an electrical power source configured to apply a current to the spring 44 via an electrical circuit. The driving element may be positioned entirely outside of the drug container 20 (e.g., and heat through the wall of the drug container 20) and/or may include a connecting element which enters the drug container 20 (e.g., to complete an electric circuit).

The driving element 40 is operably connected to the electronics 26 such that electronics 26 are configured to provide an input signal to the driving element 40. The driving element 40 may provide an input parameter to the spring 44 based on the input signal received from the electronics 26. In this way, the electronics 26 are configured to control the extension and/or retraction of the spring 44.

The drug container 20 includes a first longitudinal end 46, a second longitudinal end 48, and a plunger 50. In an exemplary embodiment, the first longitudinal end 46 is adjacent to the driving element 40 and the second longitudinal end is positioned adjacent to the fluid path 22. The plunger 50 is positioned inside of the drug container 20 and is configured to move the pharmaceutical product out of the drug container 20 via movement thereof. The spring 44 is configured to move the plunger 50. The plunger 50 is preferably sized to create a sealed arrangement inside of the drug container 20, much like a typical syringe plunger. The plunger 50 is disc-shaped or otherwise shaped to match the drug container 20.

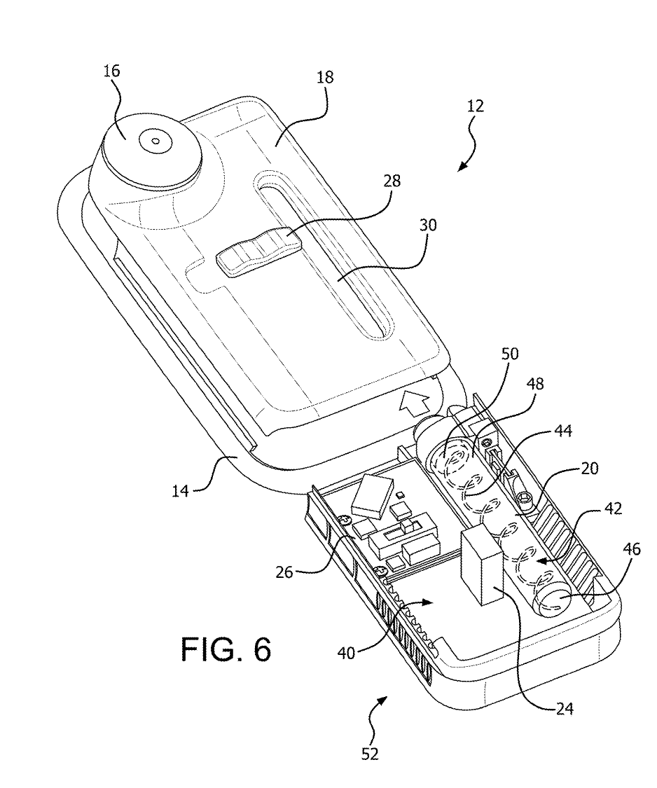

FIG. 6 illustrates an embodiment of the device 12 which includes drug container 20, drive system 24, and electronics 26 as a removable cartridge 52 relative to the housing 18, the fluid path 22, and the insertion needle 16. The drug container 20 is removable from the cartridge 52 for replacement after use. This configuration allows for insertion and replacement of the drug container 20 and helps with separating sterile components (e.g., the housing 18 and the drug container 20) from non-sterile components (e.g., the cartridge 52).

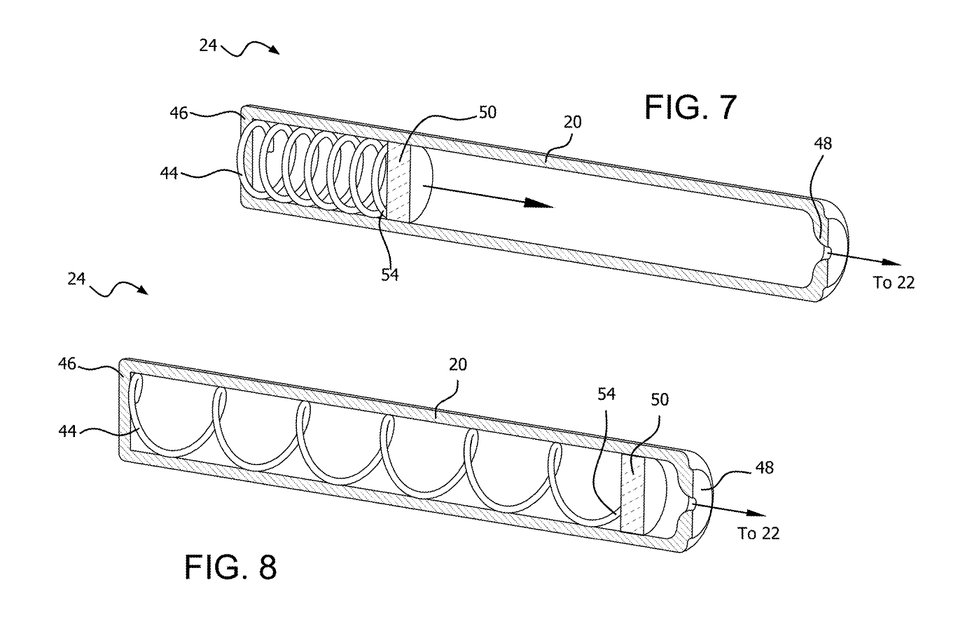

FIGS. 7 and 8 illustrate the function of the drive system 24 in relation to the drug container 20 according to a first embodiment in which the coil spring 44 is positioned inside of the drug container 20. A first end 54 of the spring 44 is in contact with the plunger 50. In FIG. 7, the spring 44 is in a retracted position. In embodiments which include only the spring 44, the spring 44 is preferably pre-configured to move to an extended position of FIG. 8, in a manner similar to a conventional spring. However, because the spring 44 is a shape memory alloy, the extension is not merely a result of a spring force, but also due to shape memory properties which cause the spring 44 to change back to its extended shape. This shape-returning force allows the spring 44 to provide a constant force which is applied to the plunger 50. In this way, the spring 44 can be configured to move the plunger 50 at a predetermined, constant rate to force the pharmaceutical product out of the drug container 20.

When included, the driving element 40 is configured to apply an input parameter to the spring 44 to change the shape of the spring 44 such that it extends to the position of FIG. 8. In the process of changing its shape to the extended position, the spring 44 pushes the plunger 50 toward the second longitudinal end 48 of the drug container 20, thereby forcing the pharmaceutical product into the fluid path 22 and ultimately delivering it to the patient 10 as needed.

While the spring 44 is illustrated and described as a coil spring, it should be understood that other configurations are possible. For example, the spring 44 may be a linear rod formed from a shape memory alloy and which is configured to extend and/or retract upon receipt of an input parameter from the driving element 40. In general, the spring 44 is configured to change in shape to linearly move the plunger 50. The change includes a change in shape due to shape memory properties. In some instances, the change in shape may be in response to receiving an input parameter from the driving element 40. Other shapes and types of springs are possible.

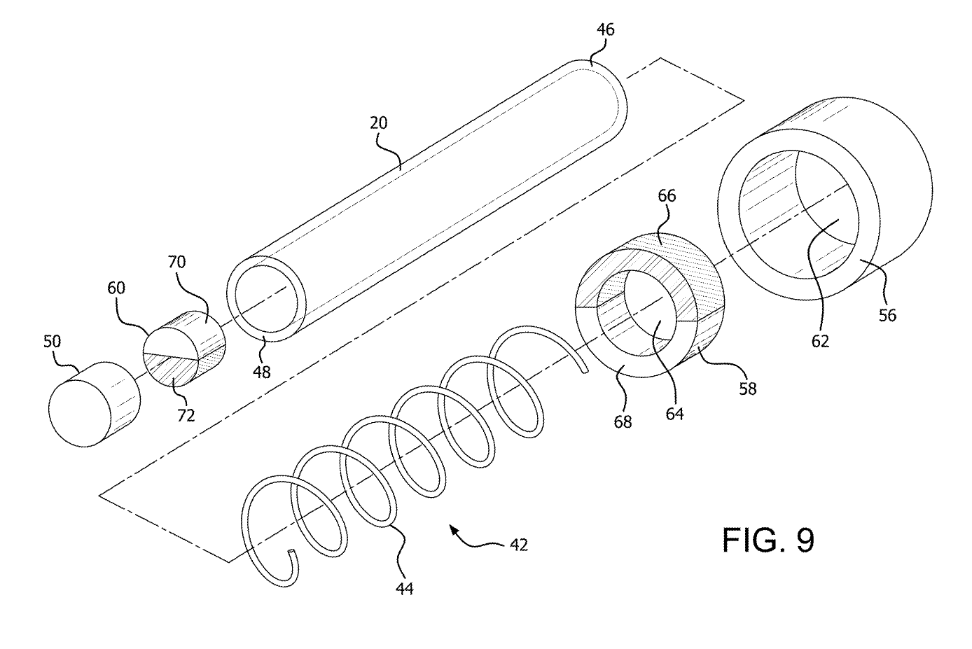

FIG. 9 is an exploded view of the movable element 42 of the drive system 24 according to another embodiment. This embodiment includes the spring 44 arranged on an exterior of the drug container 20. As shown, the movable element 42 further includes an outer collar 56, an outer magnet 58, and an inner magnet 60. The outer collar 56 is a generally cylindrical ring which includes a through-hole 62 sized to receive the drug container 20. The outer collar 56 may be generally formed of a soft magnetic alloy. In an exemplary embodiment, the outer collar 56 is in contact with the first end 54 of the spring 44. Extension and retraction of the spring 44 thus causes corresponding linear movement of the outer collar 56 along an axis of the drug container 20.

The movable element 42 further includes the outer magnet 58 and inner magnet 60 which translates movement of the outer collar 54 into movement of the plunger 50. The outer magnet 58 is a generally cylindrical ring including a through-hole 64. The outer magnet 58 is positioned in the through-hole 62 of the outer collar 56 and surrounds the exterior of the drug container 20. The outer collar 56 and outer magnet 58 may be attached to each other, such as through magnetic attraction, friction fit, adhesive, fasteners, etc. In an alternative embodiment, the outer collar 56 and the outer magnet 58 may be the same component (e.g., the outer collar 56 is diametrically magnetized or includes a magnetized portion).

The inner magnet 60 is generally cylindrical and may be solid or in the form of a ring. Other shapes of the inner magnet 60 are also possible (e.g., U-shaped, spherical, square, etc.) The inner magnet 60 is sized to fit within the drug container 20 and abuts a first side of the plunger 50. In an alternative embodiment, the inner magnet 60 and the plunger 50 are the same component (e.g., the plunger 58 is diametrically magnetized or includes a magnetized portion.

The outer magnet 58 and inner magnet 60 are configured to create a magnetic field which maintains a relative position between the two. For example, the outer magnet 58 may be diametrically magnetized with a first radial side 66 of the outer magnet 58 being a first pole and a second radial side 68 of the outer magnet being a second pole. The inner magnet 60 may be diametrically magnetized in a direction opposite from the outer magnet 58. For example, the inner magnet 60 may include a first radial side 70 which is aligned with the first radial side 66 of the outer magnet 58 and which is an opposite pole of the first radial side 66 of the outer magnet 58. Similarly, the inner magnet 60 may include a second radial side 72 which is aligned with the second radial side 68 of the outer magnet 58 and which is an opposite pole of the second radial side 68 of the outer magnet 58. In this way, the first side 66 of the outer magnet 58 is attracted to the first side 70 of the inner magnet 60 and the second side 68 of the outer magnet 58 is attracted to the second side 72 of the inner magnet 60. With this configuration, the inner magnet 60 can be positioned in the through-hole 62 of the outer magnet 58 in equilibrium such that inner magnet 60 will follow movement of the outer magnet 58.

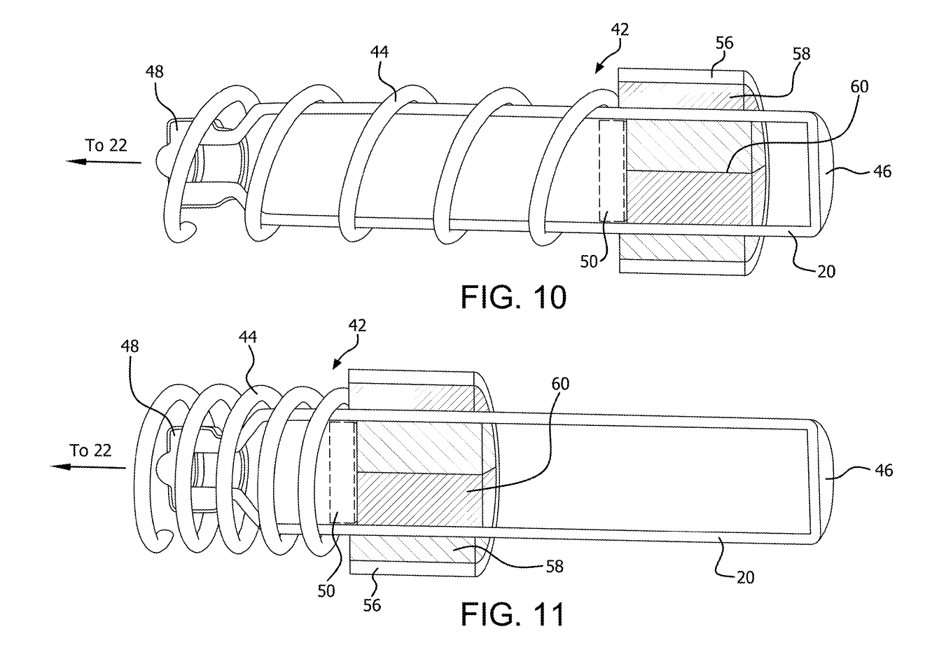

FIGS. 10 and 11 further illustrate the functioning of the drive system 24 in relation to the drug container 20 according to the embodiment in which the spring 44 is positioned on an outside of the drug container 20. As shown, the inner magnet 60 is positioned in the drug container 20 and abuts the plunger 50 (or acts as the plunger is alternative embodiments). The outer magnet 58 is positioned around the exterior of the drug container 20, in alignment with the inner magnet 60. In some embodiments, two outer magnet 58 and two inner magnets 60 may be stacked in a longitudinal direction to establish a sufficient magnetic force between the two. The outer collar 56 surrounds the outer magnet 58 (or is the outer magnet 58 in alternative embodiments). The spring 44 surrounds the drug container 20. The spring 44 may be positioned on either side of the outer collar 56. In the illustrated embodiment, the spring 44 is on a side of the outer collar 56 which faces the second longitudinal end 46.

In use, the driving element 40 is configured to provide an input parameter to the spring 44 which changes the shape of the spring 44. As described herein, the input parameter may be application of heat or current, but is not limited thereto. For example, the input parameter may be removal of heat (e.g., cooling), application of stress, etc. In the illustrated embodiment the spring 44 is initially in an extended position (FIG. 10) and application of the input parameter causes the spring to retract, pulling the outer collar 56 as the first end 54 of the spring moves toward the second longitudinal end 46 of the drug container 20 toward a retracted position (FIG. 11). In other embodiments, the configuration may be reversed such that application of the input parameter to the spring 44 causes extension of the spring 44, which pushes the outer collar 56 toward the second longitudinal end 56 of the drug container 20.

Movement of the outer collar 56 causes corresponding linear movement of the outer magnet 58. In this way, the driving element 40 is configured to cause linear movement of the outer magnet 58 along an outside of the drug container 20, in a longitudinal direction of the drug container 20 (i.e., along a longitudinal axis of the drug container 20).

The movement of the outer magnet 58 along the outside of the drug container 20 causes corresponding movement of the inner magnet 60 inside of the drug container 20. The corresponding movement of the inner magnet 60 is linear movement along the longitudinal axis of the drug container 20. The corresponding movement is enabled by the magnetic attraction between the inner magnet 60 and the outer magnet 58, which penetrates through the surface of the drug container 20.

The drive element 40 is configured to move the inner magnet 60 (via the outer magnet 58, outer collar 56, and spring 44) to force the plunger 50 toward the second longitudinal end 56 of the drug container 20. This movement of the plunger 50 forces the pharmaceutical product in the drug container out of an opening near the second longitudinal end 56 and into the fluid path 22 of the device 12. The pharmaceutical product subsequently flows through the flow path 22 and into the patient 10 through the insertion needle 16.

In embodiments which include a driving element 40, the electronics 26 are configured to calibrate application of the input parameter to the spring 44 (e.g., based on the material of the spring 44, size of the drug container 20, the viscosity of the pharmaceutical product, etc.) such that precise control over movement of the plunger 50 is possible. In this way, the driving element 40 controls (via control signals from the electronics 26) an amount, timing, and speed of an automatic injection of a pharmaceutical product from the drug container 20 into the patient 10. When the driving element 40 is omitted, the spring 44 is pre-configured to match a desired amount, timing, and speed of automatic injection. For example, the spring 44 may formed from a particular shape memory alloy material, formed in a particular shape, and/or modified with an input parameter (e.g., application of heat or current) such that the spring 44 possesses desired properties for automatically changing shape (e.g., returning to an extended shape after being compressed). The device may include a mechanical stop mechanism (not shown) which holds the spring 44 in place and which releases the spring 44 to allow for further movement.

Consistent with disclosed embodiments, the drive system 24 causes linear movement of the plunger 50 by changing the shape of the spring 44, which is formed of a shape memory alloy. In some embodiments, the spring 44 directly moves the plunger 50 (e.g., the spring 44 is arranged inside the drug container 20). In other embodiments, the spring 44 indirectly moves the plunger 50 (e.g., the spring is arranged outside the drug container 20 and is configured to move the plunger 50). For example, the driving element may be configured to cause linear movement of the outer magnet 58 which causes linear movement of the inner magnet 60 and plunger 50.

In some embodiments, the drive system 24 is configured to move the plunger 50 without breaking a barrier into the drug container 20. In the arrangement of FIGS. 7-8, the spring 44 may be a passive device which applies a constant force due to its shape memory and shape properties. In other embodiments, the driving element 40 may apply an input parameter to a shape memory alloy inside the drug container 20 without entering the drug container. For example, heat can be applied through the exterior of the drug container. In the magnetic arrangement of FIGS. 9-11, movement of a component outside of the drug container (e.g., the outer magnet) causes corresponding movement of a component inside the drug container (e.g., the inner magnet) without physically breaking a barrier into the drug container. This features is advantageous in that it helps to promote efficient use of space by omitting the need for a drive element which enters the drug container and also helps to keep sterilized and non-sterilized components separated from each other.

Some or all of the described components may be omitted and/or substituted by similar components. For example, the driving element 40 may directly move the outer magnet 58 via the spring 44, which moves the plunger 56 via the inner magnet 60. In some embodiments, the inner magnet 60 is configured as the plunger 56 such that movement of the inner magnet 60 directly forces the pharmaceutical product out of the drug container 20.

The drug container 20 may be a single-use component which is replaced after use. For example, an empty drug container 20 may be removed from the cartridge 52 (FIG. 6) and replaced with a full drug container 20. Each drug container 20 may be manufactured for use with a selected device configuration. For example, each drug container 20 may include the spring 44 or inner magnet 60 already inside the drug container 20. In other embodiments, the spring 44 or inner magnet 60 may be added to the drug container 20 before, during, or after loading into the cartridge 52.

The spring 44 may be a reusable component which is secured in place on the device 12. After an empty drug container 20 is removed, the spring 44 may be reset, either by manually reshaping the spring 44 and/or by applying another input parameter which reverses the shape of the spring 44 (e.g., extends or retracts the spring 44 back to an initial position).

In an assembly process, the drug container 20 may be slid into contact with the first end 54 of the spring 44 and or moved into the outer collar 48/outer magnet 58 elements in the cartridge 52 and then the cartridge 52 inserted into the housing 18 of the device 12. It should be understood, however, that this is an exemplary configuration and that other embodiments are possible. For example, the housing 18 may be a single unit which includes an opening for receiving the drug container 20.

The disclosed features are applicable to any injection device in order to cause movement of a plunger. This disclosed configurations are especially applicable to an automatic injection device where a driving element is present. The feature of the movable element including a shape memory alloy provides a large amount of force in a small form factor which enables a compact device. Moreover, in some embodiments, the drive system can move the plunger without physically entering the drug container.

Having thus described the presently preferred embodiments in detail, it is to be appreciated and will be apparent to those skilled in the art that many physical changes, only a few of which are exemplified in the detailed description of the invention, could be made without altering the inventive concepts and principles embodied therein. It is also to be appreciated that numerous embodiments incorporating only part of the preferred embodiment are possible which do not alter, with respect to those parts, the inventive concepts and principles embodied therein. The present embodiments and optional configurations are therefore to be considered in all respects as exemplary and/or illustrative and not restrictive, the scope of the invention being indicated by the appended claims rather than by the foregoing description, and all alternate embodiments and changes to this embodiment which come within the meaning and range of equivalency of said claims are therefore to be embraced therein.

What is claimed is:

1. An automatic injection device, comprising:
an insertion needle configured to be inserted into a patient;
a drug container which contains a pharmaceutical product, the drug container including a plunger;
a fluid path which fluidly connects the drug container to the insertion needle; and
a drive system configured to cause linear movement of the plunger to force the pharmaceutical product into the fluid path, the drive system comprising a movable element,
wherein the movable element comprises a shape memory alloy and is configured to change shape to move the plunger.

2. The automatic injection device of claim 1, wherein the movable element is a coil spring which is made of the shape memory alloy.

3. The automatic injection device of claim 2, wherein the coil spring changes shape by extending or contracting in a direction of movement of the plunger.

4. The automatic injection device of claim 3, wherein the coil spring is positioned in the drug container and includes a first end which is connected to the plunger.

5. The automatic injection device of claim 3, wherein the coil spring is positioned outside of the drug container and is configured to move the plunger indirectly.

6. The automatic injection device of claim 5, wherein the movable element further comprises an outer magnet outside of the drug container and an inner magnet inside of the drug container.

7. The automatic injection device of claim 6, wherein the spring is configured to extend or contract to move the outer magnet along the drug container to thereby move the plunger in the drug container via the inner magnet.

8. The automatic injection device of claim 7, wherein the outer magnet is diametrically magnetized in a first direction and the inner magnet is diametrically magnetized in an opposite second direction such that the inner magnet follows linear movement of the outer magnet.

9. The automatic injection device of claim 1, further including a driving element positioned outside of the drug container and configured to apply an input parameter to the movable element to change the shape of the movable element.

10. The automatic injection device of claim 9, wherein the input parameter is heating or cooling applied by the driving element to increase or decrease the temperature of the shape memory alloy.

11. The automatic injection device of claim 9, wherein the input parameter is current applied by the driving element to the shape memory alloy.

12. A product for use in an automatic injection device, the product comprising:
a drug container configured to contain a pharmaceutical product and including a first longitudinal end and a second longitudinal end;
a plunger in the drug container configured to move in a linear direction from the first longitudinal end toward the second longitudinal end; and
a movable element formed of a shape memory alloy in the drug container configured to move the plunger in the linear direction based on the shape memory properties of the movable element.

13. The product of claim 12, wherein the movable element is a coil spring made of the shape memory alloy.

14. The product of claim 12, wherein the shape memory properties include the coil spring returning to an extended shape after being compressed.

15. A cartridge for an automatic injection device, comprising:
a space for receiving a drug container which contains a pharmaceutical product; and
a drive system comprising a movable element, the movable element comprising a coil spring made from a shape memory alloy and being configured to linearly extend or contract based on the shape memory properties of the movable element in the space for receiving the drug container.

16. The cartridge of claim 15, wherein the movable element further includes an outer magnet which is configured to be moved linearly in the space for receiving the drug container by the coil spring.

17. The cartridge of claim 16, wherein the outer magnet is ring-shaped and includes a through-hole for receiving the drug container.

18. The cartridge of claim 17, wherein the movable element further includes an outer collar which includes a through-hole which receives the outer magnet.

19. The cartridge of claim 17, wherein the movable element further includes an inner magnet configured to be inserted into the drug container.

20. The cartridge of claim 15, further comprising a drive system configured to apply an input parameter to the coil spring.
